(19) United States
(12) Patent Application Publication
(10) Pub. No.: US 2025/0123295 A1
(43) Pub. Date: Apr. 17, 2025

(54) BIOMARKERS FOR EARLY DETECTION OF DIABETES

(71) Applicant: GATC HEALTH CORP, Irvine, CA (US)

(72) Inventors: Jonathan LAKEY, Irvine, CA (US); Eric J. MATHUR, Encinitas, CA (US); Ian JENKINS, Irvine, CA (US)

(73) Assignee: GATC HEALTH CORP, Irvine, CA (US)

(21) Appl. No.: 18/834,221

(22) PCT Filed: Jan. 28, 2023
(86) PCT No.: PCT/US2023/061520

§ 371 Date: Jul. 29, 2024

Related U.S. Application Data
(60) Provisional application No. 63/304,562, filed on Jan. 28, 2022.

(51) Int. Cl. G01N 33/68; G01N 33/72

(52) U.S. Cl. CPC G01N 33/6893; G01N 33/723; G01N 2333/4737; G01N 2333/54; G01N 2333/62; G01N 2800/042; G01N 2800/50

(57) ABSTRACT

Applicants have identified biomarkers that can be expressed at elevated levels in subjects who are prone to developing diabetes. The biomarkers can include misfolded proinsulin, correctly folded proinsulin, follistatin, hemoglobin A1c, C-Reactive Protein, Interleukin 18 and Interleukin 1 Receptor Antagonist, glutamic decarboxylase autoantibodies, islet cell autoantibodies, insulin autoantibodies, zinc transporter protein autoantibodies, insulinoma associated-2 autoantibodies, C-reactive protein, protectin D1, a lipoxin and a maresin. Embodiments include methods of predicting or assessing a subject's risk of developing diabetes before onset of the disease. The methods can also determine the progression and/or prognosis of diabetes. A subject who is at risk of developing the disease can be any of Type 1, Type 1.5 or Type 2 diabetes.

### RELATED APPLICATIONS

This is application claims priority to U.S. provisional patent application Ser. No. 63/304,562, filed on Jan. 28, 2022, the contents of which are incorporated herein by reference.

### FIELD OF THE INVENTION

The invention relates to clinical diagnostics, and more specifically, it relates to biomarkers and assays for early detection of diabetes.

### BACKGROUND

Diabetes mellitus (“diabetes”) refers to a group of metabolic disorders characterized by high blood sugar levels. High blood sugar levels can lead to frequent urination, increased thirst and increased appetite. If untreated, elevated sugar levels from diabetes cause many health issues. Acute complications can include diabetic ketoacidosis, hyperosmolar hyperglycemic state and death. Serious long-term complications include cardiovascular disease, kidney disease, ulcers, nerve damage, eye damage and cognitive impairment.

Diabetes is caused by either the pancreas not producing enough insulin (Type 1), or the cells of the body not responding properly to the insulin produced (Type 2). Type 1 diabetes is often referred to as “insulin-dependent diabetes” or “juvenile diabetes” and results from failure of the pancreas to produce enough insulin due to loss of beta cells. The loss of beta cells is caused by an autoimmune response. Type 2 diabetes is often referred to as “non-insulin-dependent diabetes” or “adult-onset diabetes.” It begins when the body fails to properly respond to insulin (i.e., insulin resistance). As the disease progresses, a lack of insulin production may also develop. The most common cause is a combination of excessive body weight and insufficient exercise. Gestational diabetes is the third type and occurs when pregnant women develop high blood sugar levels.

Type 1 (T1) diabetes can be considered an autoimmune disorder because insulin-producing pancreatic beta cells are targeted by an individual's own immune system. The result is a progressive destruction of beta cells, an inability to produce insulin, and chronic high blood sugar until a diagnosis is confirmed and the disease is managed with daily insulin injections. The beta cell destruction process typically begins years before the onset of symptoms, and this typically goes unnoticed as the early stages are asymptomatic. Approximately 1.6 million Americans live with Type 1 diabetes and this number is expected to increase to five million by 2050. The disease is not preventable and there is no known cure. Approximately 64,000 people are diagnosed each year contributing to the $16 billion spent annually for related healthcare costs in the U.S.

Common symptoms of T1 diabetes include unexplained increased thirst, urination, hunger, fatigue and weight loss. A random plasma glucose (RPG) test can be used as a screening tool. This test measures blood glucose (BG) at a single point in time. A resulting blood sugar value greater than 200 mg/dL suggests diabetes. As a follow-up, a fasting blood glucose (FBG) test can be performed wherein blood sugar is tested after an overnight fast. A physician may also consider levels of glycated hemoglobin (A1C) which provides an average blood glucose level for the previous three months. Generally, an AlC greater than or equal to 6.5% indicates diabetes.

Type 2 (T2) diabetes makes up about 90% of cases of diabetes. Unlike type 1 diabetes, type 2 diabetes can sometimes be prevented. Excessive weight gain, obesity and a sedentary lifestyle are known contributory factors. Rates of type 2 diabetes have increased significantly in recent decades with higher rates of obesity. As of 2015 there were approximately 392 million people diagnosed with the disease compared to around 30 million in 1985. In past decades, type 2 diabetes usually occurred only in adults. However, more kids and teens are being diagnosed with type 2 diabetes due to the increasing rates of obesity. Type 2 diabetes is largely preventable by maintaining a normal weight, exercising regularly and eating a healthy diet. Diagnosis of Type 2 diabetes is associated with a ten-year-shorter life expectancy.

Globally, an estimated 462 million people are affected by type 2 diabetes, corresponding to 6.28% of the world's population. More than one million deaths were attributed to this condition in 2017, ranking it as the ninth leading cause of mortality. In the U.S., approximately 25 million Americans ages 20-64 have diabetes which corresponds to about 11% of this age group. Total health care and related costs are approximately $174 billion annually.

An astounding 86 million people in the U.S. have prediabetes, a condition marked by elevated blood sugar that gives rise to type 2 diabetes. Of these individuals, 90% do not know that they have the condition which increases their risk of developing heart disease and stroke. When an individual has prediabetes, the increased sugar in their blood triggers the body to produce larger amounts of insulin. However, full blown diabetes can occur if the beta cells in their pancreas fail to keep up with the demand. In both type 1 and type 2 diabetes, various genetic and environmental factors can result in the progressive loss of β-cell mass and/or function that manifests clinically as hyperglycemia. Once hyperglycemia occurs, patients with all forms of diabetes are at risk for developing the same chronic complications, although rates of progression may differ. Accordingly, the identification of individualized therapies for diabetes requires better characterization of the many paths to β-cell demise or dysfunction.

The most common test for diagnosing T2 diabetes is a blood test that compares the amount of glycated hemoglobin to non-glycated hemoglobin (i.e., AlC test). Glycation of proteins is a frequent occurrence, but in the case of hemoglobin, a nonenzymatic condensation reaction occurs between glucose and the N-end of the beta chain. This reaction produces a Schiff base, which is itself converted to 1-deoxyfructose. This second conversion is an example of an Amadori rearrangement. When blood glucose levels are high, glucose molecules attach to the hemoglobin in red blood cells. The longer hyperglycemia occurs in blood, the more glucose binds to hemoglobin in the red blood cells and the higher the glycated hemoglobin.

After a hemoglobin molecule is glycated, it remains that way. Therefore, a buildup of glycated hemoglobin within the red cell reflects the average level of glucose to which the cell has been exposed during its lifecycle. Measuring glycated hemoglobin assesses the effectiveness of therapy by monitoring long-term serum glucose regulation. Hemoglobin A1C tests involve two measurements: the measurement of hemoglobin A1c and the measurement of total hemoglobin. The ratio of A1c to total hemoglobin is an assessment of the degree of glycation. It is typically reported as a percentage such that a healthy level is less than 5.7%.

The ratio of glycated hemoglobin (A1c) is widely accepted as a valuable marker of long-term glycemic control. A1c provides valuable information for evaluating average blood glucose levels over a period of two to three months. It has particular clinical utility because levels are minimally affected by short, large fluctuations in blood glucose concentration. A1c levels are also an important tool in evaluating the effectiveness of new glycemic control regimens.

A1c is measured primarily to determine the three-month average blood sugar level and can be used as a diagnostic test for diabetes mellitus and as an assessment test for glycemic control in people with diabetes. The test is limited to a three-month average because the average lifespan of a red blood cell is four months. Additionally, due to individual red blood cells have varying lifespans, the test is typically administered in three-month intervals. Normal levels of glucose produce a normal amount of glycated hemoglobin. As the average amount of plasma glucose increases, the fraction of glycated hemoglobin predictably increases.

Unfortunately, an elevated A1c can indicate that damage has already occurred. Glycated hemoglobin causes an increase of highly reactive free radicals inside blood cells. Radicals alter blood cell membrane properties. This leads to blood cell aggregation and increased blood viscosity, which results in impaired blood flow. Another way glycated hemoglobin causes damage is via inflammation, which results in atherosclerotic plaque (atheroma) formation. Free-radical build-up promotes the excitation of Fe2+-hemoglobin through Fe3+-Hb into abnormal ferryl hemoglobin (Fe4+-Hb). Fe4+ is unstable and reacts with specific amino acids in hemoglobin to regain its Fe3+ oxidation state. Hemoglobin molecules clump together via cross-linking reactions, and these hemoglobin clumps (multimers) promote cell damage and the release of Fe4+-hemoglobin into the matrix of innermost layers (i.e., subendothelium) of arteries and veins. This results in increased permeability of interior surface (i.e., endothelium) of blood vessels and production of pro-inflammatory monocyte adhesion proteins which promote macrophage accumulation in blood vessel surfaces, ultimately leading to harmful plaques in these vessels.

Highly glycated Hb-AGEs go through vascular smooth muscle layer and inactivate acetylcholine-induced endothelium-dependent relaxation, possibly through binding to nitric oxide (NO), preventing its normal function. NO is a potent vasodilator that also inhibits formation of plaque-promoting LDLs (i.e., “bad cholesterol”) oxidized form. This overall degradation of blood cells also releases heme from them. Loose heme can cause oxidation of endothelial and LDL proteins which results in plaque. Because of these harmful effects, it would be beneficial to recognize a patient's likelihood of developing diabetes before levels of glycated hemoglobin increase.

T2 diabetes develops slowly. Typically, people first become aware of the disease either because of another condition or by a blood test performed as part of a routine checkup. In some cases, T2 diabetes is not detected until damage to the eye, kidney or other organs occurs. Because of the progressive nature of T2 diabetes, early efforts to recognize risk factors and diagnose it in early stages can improve long term outcomes. Accordingly, there is a need for improved methods of identifying individuals at risk of developing T2 diabetes.

An improved diagnostic test for diabetes should allow a healthcare provider to predict one's predisposition to T2 diabetes. Such a test could allow patients and providers to take efforts to prevent/ameliorate the disease before blood sugar levels are elevated. It would be particularly useful to have a method of predicting or assessing an individual's risk of developing diabetes before the onset of the disease. Doing so could help prevent damage to one's organs and circulatory system. Applicants have identified a set of biomarkers that can be measured to identify whether the patient is at risk of developing diabetes. The biomarkers can also be used to monitor the progress of T2 diabetes. The present invention also includes methods and an assay/kit for quantifying levels of the biomarkers.

### SUMMARY OF THE INVENTION

The inventions described and claimed herein have many attributes and embodiments including those set forth or described or referenced in this brief summary. The inventions described and claimed herein are not limited to, or by, the features or embodiments identified in this summary, which is included for purposes of illustration only and not restriction.

As described herein, Applicants have identified biomarkers for identifying one's risk of developing T2 diabetes. Specifically, Applicants identified proteins that are upregulated in subjects who are susceptible to developing diabetes. In one embodiment, the biomarkers are misfolded proinsulin, correctly folded proinsulin, follistatin, hemoglobin A1c, C-Reactive Protein, Interleukin 18 and Interleukin 1 Receptor Antagonist. In aspects, the diabetes is characterized as prediabetes, metabolic syndrome, insulin resistance, glucose intolerance, glucose non-responsiveness, T1 diabetes or T2 diabetes.

Embodiments include the use of two or more biomarkers for early detection of T2 diabetes in a subject. The biomarkers are particularly useful because they can indicate that a subject is at risk of developing diabetes before damage occurs. Therapies can be implemented to prevent/ameliorate the onset of diabetes as well as prevent damage that occurs from elevated blood sugar levels.

Accordingly, embodiments also include the use of two or more biomarkers for determining the progression (or regression) of diabetes in a subject.

Embodiments also include the use of hemoglobin A1c and one or more additional biomarkers for early detection of diabetes in a subject. Embodiments also include the use of hemoglobin A1c and one or more additional biomarkers for determining the progression (or regression) of diabetes in a subject.

Embodiments also include a method and assay that uses two or more biomarkers for predicting diabetes in a subject. Embodiments also include a method and assay that uses two or more biomarkers for determining the progression of diabetes in a subject (e.g., T2 diabetes).

Embodiments also include methods of preventing diabetes by monitoring changes of levels for one or more biomarkers in a sample from a subject. The biomarkers can include one or more of this listed herein (e.g., FIG. 5).

Embodiments also include methods of treating diabetes (including predisposition to diabetes) by monitoring progression of diabetes in a subject.

Embodiments also include proteins (i.e., biomarkers) and methods for early detection of an ailment in a subject. The ailment can be insulin resistance, glucose intolerance, glucose non-responsiveness and/or diabetes (T1 or T2). In one embodiment, the biomarkers include one or more of misfolded proinsulin, correctly folded proinsulin, follistatin, hemoglobin A1c, C-Reactive Protein, Interleukin 18, and Interleukin 1 Receptor Antagonist. In one embodiment, the biomarkers include one or more of protectin D1, a lipoxin and a maresin. In one embodiment, the biomarkers include one or more of glutamic decarboxylase autoantibodies, islet cell autoantibodies, insulin autoantibodies, zinc transporter protein autoantibodies, insulinoma associated-2 autoantibodies and C-reactive protein. In one embodiment, the biomarkers are hemoglobin A1c and fructosamine/glycated albumin.

Embodiments also include diagnostic biomarkers for early identification of pre-diabetic candidates. The biomarkers can identify and determine the risk of developing diabetes in subjects with pre-diabetes, monitor disease onset and progression and/or regression. Further, the biomarkers can be used to develop therapies and test therapies in patients with pre-diabetes and/or diabetes.

Embodiments also include a method of determining a subject's susceptibility to the development of T2 diabetes. The method can include a step of analyzing a sample from the subject to determine the level of two or more biomarkers. The biomarkers can be two or more of misfolded proinsulin, correctly folded proinsulin, follistatin, hemoglobin A1c, C-Reactive Protein, Interleukin 18, and Interleukin 1 Receptor Antagonist. In one embodiment, the biomarkers are hemoglobin A1c and fructosamine/glycated albumin.

The level of biomarkers can be used to determine whether the subject is susceptible to the development of type 2 diabetes. The method can also include a step of treating the subject (e.g., by exercising, losing weight, maintaining a healthy body mass index (BMI), dieting or taking medication).

Embodiments also include a method of monitoring the progression or regression of pre-diabetes in a subject. The method can include a step of analyzing a sample from the subject to determine the level of two or more biomarkers (listed in FIG. 5). The level of biomarkers can be obtained over time to monitor the progression and/or regression of pre-diabetes. The method can also include a step of treating the subject. In an embodiment, the method includes analysis of additional biomedical information from the subject.

Embodiments also include a method of identifying/diagnosing diabetes or determining a prognosis of a subject with diabetes. The method can include steps of (a) measuring expression levels of at two least protein, peptide or lipid biomarkers in a test sample from the subject, (b) receiving the expression levels with a computer, (c) compiling the expression levels to yield a score, and (d) comparing the score to two or more threshold values to diagnose or determine the prognosis of diabetes. The method can also be used to monitor progression (or regression) of diabetes.

Embodiments also include a method of identifying/diagnosing an ailment (i.e., diabetes) or determining a prognosis of a test subject with the ailment. The method can include steps of: (a) measuring expression levels of two or more biomarkers from subjects with the ailment; (b) measuring expression levels of the two or more biomarkers obtained from healthy subjects; (c) comparing the expression levels of the two or more biomarkers from samples from the subjects with the ailment to the levels in samples from the healthy subjects; (d) identifying biomarkers that have altered levels of expression obtained from blood samples from the subjects with the ailment; (e) creating a biomarker fingerprint from the biomarkers with altered levels of expression; and (f) diagnosing or determining the prognosis of the ailment in the test subject by comparing of levels of biomarkers of the test subject to those in the biomarker fingerprint. The method can also include a step of treating the subject to prevent/ameliorate the onset of diabetes and/or prevent damage that occurs from elevated blood sugar levels/elevated A1C. The method can use biomarkers identified herein (e.g., FIG. 5). In aspects, the ailment is prediabetes, metabolic syndrome, insulin resistance, glucose intolerance, glucose non-responsiveness, T1 diabetes and/or T2 diabetes.

Embodiments also include a method of identifying/diagnosing an ailment (i.e., diabetes) or determining a prognosis. The method can include detecting changes in the ratio between fructosamine (i.e., glycated albumin) and HbA1C (i.e., glycated hemoglobin). Alternatively, the method can include detecting changes in the ratio between glycated albumin and HbA1C. In one aspect, the method can provide a patient with a one-year advanced notice predisposition for T2 diabetes.

Embodiments also include a method of monitoring the progression of an ailment such as diabetes. The method can include detecting changes in the ratio between fructosamine (i.e., glycated albumin) and HbA1C (i.e., glycated hemoglobin). Alternatively, the method can include detecting changes in the ratio between glycated albumin and HbA1C.

Embodiments also include a method of predicting, identifying or diagnosing an ailment (i.e., diabetes) or determining a prognosis. The method can include detecting changes in fructosamine, glycated albumin and/or HbA1C (i.e., glycated hemoglobin) over a period of time (e.g., six months, one year or two years). Alternatively, the method can include detecting changes from a first time (t₁) to a second time (t₂). The ratio (or percentage increase) over time or between the time points can be used to determine a likelihood of developing diabetes. The method can also be used to monitor the progression of an ailment such as diabetes.

Embodiments also include a method of identifying/diagnosing an ailment (i.e., T2 diabetes) or determining a prognosis. The method can include detecting changes in the ratio of lipid intermediary biomarkers. The lipid intermediary biomarkers can include protectin D1, lipoxins and maresins.

Embodiments also include a method of monitoring the progression of an ailment such as T2 diabetes. The method can include detecting changes in the ratio between lipid intermediary biomarkers. The lipid intermediary biomarkers can include protectin D1, lipoxins, and maresins.

Embodiments also include a method of identifying/diagnosing an ailment (i.e., T1 diabetes) or determining a prognosis. The method can include detecting the presence (or increased expression) of one or more autoantibodies as biomarkers. The autoantibodies can include Glutamic Decarboxylase Autoantibodies (GAD65), Islet Cell Autoantibodies (ICA), Insulin Autoantibodies (IAA), Zinc Transporter Protein Autoantibodies (ZnT8), Insulinoma Associated-2 Autoantibodies (IA-2A) and C-Reactive Protein. The method can include treating the subject by, for example, administering one or more immunomodulating agents.

Embodiments also include a method of monitoring the progression of an ailment such as T1 diabetes. The method can include detecting changes in the ratio between lipid intermediary biomarkers. The lipid intermediary biomarkers can include protectin D1, lipoxins, and maresins.

In aspects, the methods described herein can be used to identify, diagnose, determine a predisposition to and/or determine a prognosis of a subject to diabetes. The diabetes can be type 1, type 2, type 3, type 1.5, group 1, group 2, group 3, group 4 or group 5.

Embodiments also include a lateral flow assay that can detect protein biomarkers at low levels. The levels of protein can also be determined by analyzing the amount of an indicator of fluorescence.

Embodiments also include a diagnostic kit for diagnosing an ailment (i.e., insulin resistance, glucose intolerance, glucose non-responsiveness and/or type 2 diabetes mellitus). The kit can be used to detect hemoglobin A1c and one or more additional biomarkers.

Embodiments also include an assay or kit for early detection/diagnosis of T2 diabetes in a subject. The kit can use upconverting phosphor nanoparticles for the detection and/or quantification of one or more biomarkers.

Embodiments also include a rapid lateral flow (LF) based antibody screening assay. The assay can use nano-sized up-converting phosphor (UCP) reporter particles. An analyzer can detect antibodies specific to biomarkers and quantify the amount of biomarkers (i.e., misfolded proinsulin, correctly folded proinsulin, follistatin and hemoglobin A1c, C-Reactive Protein, Interleukin 18, and Interleukin 1 Receptor Antagonist) in a sample.

### BRIEF DESCRIPTION OF THE DRAWINGS

The accompanying drawings illustrate aspects of the present invention. In such drawings:

FIG. 1 is a flowchart of the steps involved in testing a patient for susceptibility or progression of Type 2 diabetes according to embodiments.

FIG. 2 is a flowchart of the steps involved in testing a patient for susceptibility or progression of Type 1 diabetes by detecting autoantibodies according to embodiments.

FIG. 3 is a flowchart of the steps involved in testing a patient for susceptibility or progression of Type 2 diabetes by analyzing levels of GAB and HbA1c according to embodiments.

FIG. 4 is a flowchart of the steps involved in testing a patient for susceptibility or progression of Type 2 diabetes by comparing ratios of lipid intermediate biomarkers according to embodiments.

FIG. 5 is a table of proteins and lipids that can be used as markers for early detection of diabetes according to embodiments.

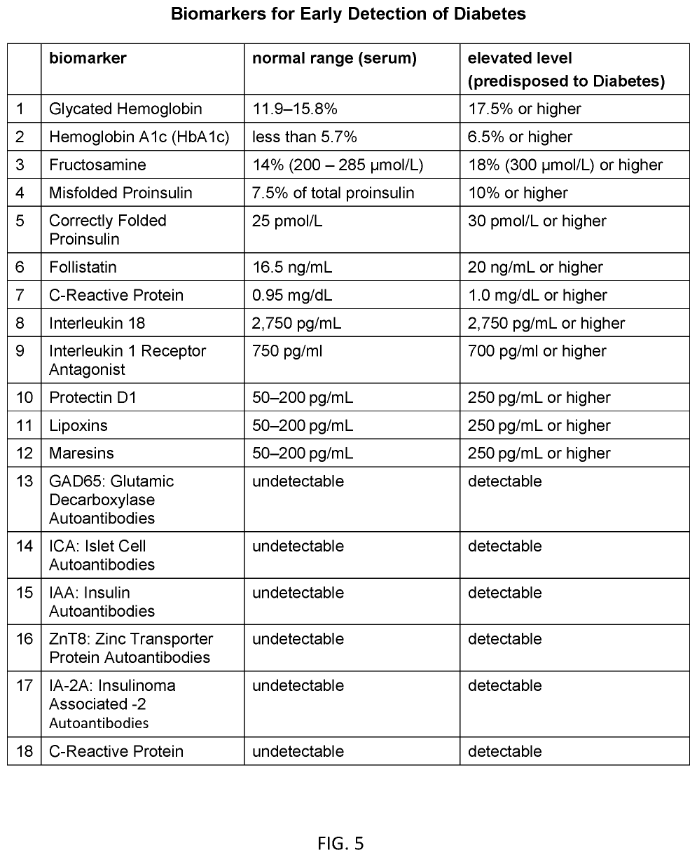

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “glucose intolerance” refers to insufficiency of an insulin secretion response due to glucose load and/or reduction of insulin action in skeletal muscles or adipose tissues. Accordingly, the subject is unable to utilize glucose in blood circulation. In some cases, glucose intolerance is caused by insulin resistance. Glucose intolerance may precede the onset of diabetes and it may be associated with various metabolic diseases or conditions, such as obesity, hypertension, hypertriglyceridemia, etc. Continuous glucose intolerant conditions may induce onset of diabetes and may also enhance the progress of diabetes. Therefore, treatment of glucose intolerance is considered effective in reducing an incidence or progression of diabetes.

The term “glucose non-responsiveness” refers to the complete inability of cells, islets or mammals to respond to treatment with or administration of glucose, as well as decreased responsiveness to glucose (e.g., by cells that do not produce sufficient levels of insulin in response to glucose or that require significantly higher levels of glucose to respond at normal levels).

The term “diabetes” or “diabetes mellitus” refers to a disease that occurs when the body cannot make use of the glucose in the blood for energy because either the pancreas is not able to make enough insulin or the insulin that is available is not effective. There are two main types of diabetes mellitus: insulin-dependent (Type 1) and noninsulin-dependent (Type 2 or adult-onset diabetes). A third type of diabetes is gestational diabetes that can develop during pregnancy. Diabetes can also lead to the development of other diseases or conditions and is a risk factor in the development of conditions such as metabolic syndrome and cardiovascular disease. Metabolic syndrome is a grouping of a series of risk factors in an individual.

Type 1.5 diabetes, also called latent autoimmune diabetes in adults (LADA), is a condition that shares characteristics of both type 1 and type 2 diabetes. LADA is diagnosed during adulthood, and it sets in gradually, like type 2 diabetes. But unlike type 2 diabetes, LADA is an autoimmune disease and is not reversible with changes in diet and lifestyle. Beta cells stop functioning more quickly with type 1.5 diabetes than type 2. It is estimated that 10 percent of people who have diabetes have LADA. Type 1.5 diabetes is often misdiagnosed as type 2 diabetes.

Recent studies have proposed five groups of diabetes based on factors such as age, body mass index, the presence of beta-cell antibodies, level of metabolic control, measures of beta-cell function and insulin resistance. These groups are:
Group 1: severe autoimmune diabetes (currently known as type 1 diabetes), characterized by insulin deficiency and the presence of autoantibodies. This was identified in 6-15 percent of subjects.
Group 2: severe insulin-deficient diabetes, characterized by younger age, insulin deficiency, and poor metabolic control, but no autoantibodies. This was identified in 9-20 percent of subjects.
Group 3: severe insulin-resistant diabetes, characterized by severe insulin resistance and a significantly higher risk of kidney disease. This was identified in 11-17 percent of subjects.
Group 4: mild obesity-related diabetes, most common in obese individuals. This affected 18-23 percent of subjects.
Group 5: mild age-related diabetes, most common in elderly individuals. This was the most common form, affecting 39-47 percent of subjects.

The term “predisposition” refers to an increased chance or likelihood of developing a particular ailment (e.g., diabetes) based on, for example, increased levels of one or more biomarkers. A predisposition can also be identified by the presence of a genetic variants and/or a family history suggestive of an increased risk of the ailment.

The term “advanced glycation end products” or “AGEs” refers to proteins or lipids that become glycated as a result of exposure to sugars. They can be used as biomarkers in the development, or worsening, of many degenerative diseases, including diabetes, atherosclerosis, chronic kidney disease and Alzheimer's disease. Accumulation of advanced glycation end products (AGEs) on nucleotides, lipids and peptides/proteins have also been studied in relation to the aging process in eukaryotic organisms. Evidence suggests that AGEs and their functionally compromised adducts are linked to and perhaps responsible for changes seen during aging and for the development of many age-related morbidities. Proteins that can become glycated from elevated glucose levels include albumin, fibrinogen, collagen and immunoglobin.

The term “metabolic syndrome” refers to a cluster of conditions that occur together, increasing the risk of heart disease, stroke and Type 2 diabetes. These conditions include increased blood pressure, high blood sugar, excess body fat around the waist, and abnormal cholesterol or triglyceride levels.

The term “prediabetes” refers to one or more early diabetic conditions, including impaired glucose utilization, fasting plasma glucose abnormalities or disorders, impaired glucose tolerance, insulin sensitivity abnormalities and insulin resistance.

The term “glycated hemoglobin,” “glycohemoglobin,” “hemoglobin A1c,” “HbA1c,” or “A1C” refers to a form of hemoglobin (Hb) that is chemically linked to a sugar. Most monosaccharides, including glucose, galactose and fructose, spontaneously bond with hemoglobin when present in the bloodstream. The formation of the sugar-hemoglobin linkage indicates the presence of excessive sugar in the bloodstream, often indicative of diabetes. A1C is of particular interest because it is easy to detect. The process by which sugars attach to hemoglobin is called glycation. HbA1c is a measure of the beta-N-1-deoxy fructosyl component of hemoglobin. Methods of measuring of A1C percentage (%) utilizing antibodies are well known in the art. Two separate measurements are used, one for the concentration of glycated hemoglobin the other for the concentration of total hemoglobin. The A1C % is calculated as a ratio of [Hemoglobin A1c]/[Hemoglobin]×100. Higher levels of HbA1c are found in people with persistently elevated blood sugar. In diabetes, higher amounts of glycated hemoglobin, indicating poorer control of blood glucose levels, have been associated with cardiovascular disease, nephropathy, neuropathy, and retinopathy.

The term “albumin” refers to a family of globular proteins, the most common of which are the serum albumins. All the proteins of the albumin family are water-soluble, moderately soluble in concentrated salt solutions and experience heat denaturation. Albumins are commonly found in blood plasma and differ from other blood proteins in that they are not glycosylated. Substances containing albumins are called albuminoids.

The term “human serum albumin” or “HSA” refers to the main protein of human blood plasma. It makes up around 50% of human plasma proteins. It binds water, cations (such as Ca²⁺, Na⁺ and K⁺), fatty acids, hormones, bilirubin, thyroxine (T4) and pharmaceuticals (including barbiturates). Its main function is to regulate the oncotic pressure of blood.

The term “fructosamine” refers to a compound that results from glycation reactions between a sugar (e.g., fructose or glucose) and a primary amine, followed by isomerization via the Amadori rearrangement. Biologically, fructosamines are recognized by fructosamine-3-kinase, which may trigger the degradation of advanced glycation end-products (though the true clinical significance of this pathway is unclear).

The term “glycated albumin” or “GA” refers to the glycated form of circulating albumin. Assays for total serum glycated proteins (i.e., fructosamine) and the more specific glycated albumin can be useful indicators of hyperglycemia. Due to the shorter lifespan of albumin in comparison to the traditional biomarkers of glycemic control (e.g., HbA1c), GA can be used as a biomarker of early response to hypoglycemic treatment.

In diabetes, blood sugars are often measured by either blood glucose monitoring which measures the current blood glucose level, or by glycated hemoglobin (i.e., HbA1c) which measures average glucose levels over approximately three months. In a similar way to hemoglobin A1c testing (which measures the glycation of hemoglobin), fructosamine testing determines the fraction of total serum proteins that have undergone glycation (i.e., the glycated serum proteins). Because albumin is the most abundant protein in blood, fructosamine levels typically reflect albumin glycation. A fructosamine test can specifically quantify the glycation of albumin, or glycated serum albumin. Because albumin has a half-life of approximately 20 days, the plasma fructosamine concentration reflects relatively recent (i.e., one to two week) changes in blood glucose.

The term “biomarker” refers generally to a DNA, RNA, protein, carbohydrate, or glycolipid-based molecular marker, the expression or presence of which in a subject's sample can be detected by standard methods (or methods disclosed herein) and is predictive or prognostic of the effective responsiveness or sensitivity of a mammalian subject with an ailment. Biomarkers may be present in a test sample but absent in a control sample, absent in a test sample but present in a control sample, or the amount of biomarker can differ between a test sample and a control sample. For example, genetic biomarkers assessed (e.g., specific mutations and/or SNPs) can be present in such a sample, but not in a control sample, or certain biomarkers are seropositive in the sample, but seronegative in a control sample. Also, optionally, expression of such a biomarker may be determined to be higher than that observed for a control sample. The expression of biomarkers typically includes both up- and down-regulated levels. However, some useful biomarkers will not have altered levels of expression. The terms “marker” and “biomarker” can be used herein interchangeably.

The amount of the biomarker can be measured in a test sample and compared to the “normal control level,” utilizing techniques such as reference limits, discrimination limits, or risk defining thresholds to define cutoff points and abnormal values for an ailment. The normal control level means the level of one or more biomarkers or combined biomarker indices typically found in a subject not suffering from an ailment (or prone to an ailment such as T2 diabetes). Such normal control level and cutoff points can vary based on whether a biomarker is used alone or in a formula combining with other biomarkers into an index. Alternatively, the normal control level can be a database of biomarker patterns from previously tested subjects who did not experience the ailment over a clinically relevant time.

After selection of a set of biomarkers, well-known techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms, or other mathematical and statistical methods can be used to develop a formula for calculation of a risk score. A selected population of individuals is used, where historical information is available regarding the values of biomarkers in the population and their clinical outcomes. To calculate a risk score for a given individual, biomarker values are obtained from one or more samples collected from the individual and used as input data.

Tests to measure biomarkers and biomarker panels can be implemented on a variety of diagnostic test systems. Diagnostic test systems are apparatuses that typically include means for obtaining test results from biological samples. Examples of such means include modules that automate the testing (e.g., biochemical, immunological, nucleic acid detection assays). Some diagnostic test systems are designed to handle multiple biological samples and can be programmed to run the same or different tests on each sample. Diagnostic test systems typically include means for collecting, storing and/or tracking test results for each sample, usually in a data structure or database. Examples include well-known physical and electronic data storage devices (e.g., hard drives, flash memory, magnetic tape, paper printouts). It is also typical for diagnostic test systems to include means for reporting test results. Examples of reporting means include visible display, a link to a data structure or database, or a printer. The reporting means can be a data link to send test results to an external device, such as a data structure, data base, visual display, or printer.

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., diabetic samples and normal or control samples). ROC curves are useful for plotting the performance of a particular feature (e.g., any of the biomarkers described herein and/or any item of additional biomedical information) in distinguishing between two populations (e.g., cases having T2 diabetes and controls without T2 diabetes). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

The term “fingerprint,” “disease fingerprint,” or “biomarker signature” refers to a plurality or pattern of biomarkers that have elevated or reduced levels in a subject with disease. A fingerprint can be generated by comparing subjects with the disease to healthy subjects and used for screening/diagnosis of the disease.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “prognosis” refers to a likely course (i.e., a forecast) of a disease or ailment such as T2 diabetes. As used herein, it refers to the probable outcome of T2 diabetes, including whether the disease will respond to treatment or mitigation efforts and/or the likelihood that the disease will progress.

As used herein, “additional biomedical information” refers to one or more evaluations of an individual, other than using any of the biomarkers described herein, that are associated with health and/or susceptibility to diabetes. Accordingly, “additional biomedical information” includes any of the following: physical descriptors of an individual, the height, weight and/or BMI of an individual, the gender of an individual, the ethnicity of an individual, family history, smoking history, occupational history, age, BMI, waist circumference, history of antihypertensive drug treatment and high blood glucose, physical activity, diet, etc. Additional biomedical information can be obtained from an individual using routine techniques known in the art, such as from the individual themselves by use of a routine patient questionnaire or health history questionnaire, etc., or from a medical practitioner, etc. Testing of biomarker levels in combination with an evaluation of any additional biomedical information may, for example, improve sensitivity, specificity, and/or AUC for predicting T2 diabetes (or other related uses) as compared to biomarker testing alone or evaluating any particular item of additional biomedical information alone.

The term “immunoassay” refers to a biochemical test that measures the presence or concentration of a substance in a sample, such as a biological sample. It is common to use the reaction of an antibody to its cognate antigen, for example the specific binding of an antibody to a protein. Both the presence of antigen and the amount of antigen present can be measured. The presence and amount (i.e., abundance) of the protein can determined or measured. Measuring the quantity of antigen (such as a biomarker) can be achieved by a variety of methods. A common method is to label either the antigen or antibody with a detectable label (e.g., a fluorescent tag, enzymatic linkage or radioactive isotope).

The term “lateral flow assay,” “lateral flow immunoassay” or “LFA” refers to a diagnostic device used to confirm the presence or absence of a target analyte. LFA-based tests often use a paper-based platform for the detection and quantification of analytes, where the sample is placed on a test device and the results are displayed within 5-30 minutes. LFA-based tests are widely used in hospitals and clinical laboratories for the qualitative and quantitative detection of specific antigens and antibodies, as well as products of gene amplification. The principle behind the LFA is relatively simple. A liquid sample (or its extract) containing the analyte of interest moves without the assistance of external forces (capillary action) through various zones of polymeric strips, on which molecules that can interact with the analyte are attached. A typical lateral flow test strip has overlapping membranes that are mounted on a backing card.

The term “target analyte” or “analyte” refers to a molecule, compound or particle to be detected. Target analytes bind to binding ligands (both capture and soluble binding ligands), as is more fully described below. In some embodiments, the target analyte is a protein such as a biomarker (e.g., proinsulin, follistatin, A1c, C-Reactive Protein, Interleukin 18, or Interleukin 1 Receptor Antagonist) as described herein.

The term “substrate” or “solid support” refers to a material that can be modified to contain discrete individual sites appropriate of the attachment or association of capture ligands. Suitable substrates include metal surfaces such as gold, electrodes as defined below, glass and modified or functionalized glass, fiberglass, resins, silica or silica-based materials, carbon, metals, inorganic glasses and other polymers.

The term “up-converting nano-phosphors” or “upconverting materials” refers to compounds that emit light at a wavelength that is shorter than the wavelength of light they have been photoexcited with which give them applications in biomedical imaging. The so-called anti-Stokes shift in these materials limits the autofluorescence of nearby molecules within a sample. Compared with gold nanoparticles, a lateral flow test using upconverting phosphor nanoparticles (UCNPs) is more sensitive (approximately tenfold) and robust, due to the unique feature of using the lower energy 980 nm infrared light (excitation light) to generate higher energy visual light (emission light). This light process is called “up-conversion,” which does not occur in nature. Thus, UCNPs as a reporter label do not generate background fluorescence (autofluorescence) compared with conventional fluorescent labels, such as fluorescently labeled nanoparticles and quantum dots. Moreover, UCNPs do not fade, allowing the lateral flow strips based on UCNPs to be stored in the long term. More importantly, the lateral flow test based on UCNPs has no interference from red blood cell hemolysis that is a problem sometimes encountered in lateral flow test based on colloidal gold-labeled nanoparticles using blood.

The term “immunomodulatory therapy” refers to a treating an ailment of or related to immune system activity. Immunomodulatory therapies have been used to treat ailments such as cancer, multiple sclerosis, Crohn's disease and influenza A virus.

The term “immunomodulating agent” refers to a substance that stimulates or suppresses the immune system and may help the body fight cancer, infection or other diseases. Specific immunomodulating agents, such as monoclonal antibodies, cytokines, and vaccines, affect specific parts of the immune system. Nonspecific immunomodulating agents, such as BCG and levamisole, affect the immune system in a general way. Other immunomodulating agents include steroids, Cyclosporine A, tacrolimus (either individually, or combined with rapamycin) or azathioprine and T cell inhibitors.

Similarly, an “immunosuppressive agent” is a substance that decreases the body's immune responses. It can reduce the body's ability to fight infections and other diseases, such as cancer. However, immunosuppressive agents can be used to treat autoimmune disorders. They can also be used to keep a person from rejecting a bone marrow/organ transplant. Common immunosuppressive agents include, for example, cyclophosphamide, rituximab, methotrexate, azathioprine or a glucocorticoid.

The term “prevention” means all of the actions by which the occurrence of the disease is restrained, ameliorated or retarded.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “sample” refers to a biological sample obtained from an individual, body fluid, body tissue, cell line, tissue culture, or other source. Body fluids are, for example, lymph, sera, whole fresh blood, peripheral blood mononuclear cells, frozen whole blood, plasma (including fresh or frozen), urine, saliva, semen, synovial fluid and spinal fluid. Samples also include synovial tissue, skin, hair follicle, and bone marrow. Methods for obtaining tissue biopsies and body fluids from mammals are well known in the art.

The term “risk score” refers to a general practice in applied statistics, biostatistics, econometrics and other related disciplines, of creating an easily calculated number that reflects the level of risk in the presence of some risk factors. Risk scores are a way of stratifying a population for targeted screening. They use data from risk factors to calculate an individual's score; a higher score reflects higher risk. Risk scores can be applied either to an individual as a questionnaire (these scores generally require only data from non-invasive risk factors, which would be known by members of the public) or to a population. The “diabetes risk score” was developed as a screening tool for identifying high-risk subjects in the population and for increasing awareness of the modifiable risk factors and healthy lifestyle. It uses a series of questions to identify risk factors in a subject.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the methods particularly pointed out in the written description and claims hereof.

In Type 1 (T1) diabetes, insulin-producing pancreatic beta cells are targeted by an individual's own immune system. This leads to high levels of blood sugar as the body in unable to produce insulin. The destruction of beta cells typically begins years before the onset of symptoms and typically goes unnoticed because the beginning stages of T1 diabetes are asymptomatic. After T1 diabetes is identified, it can be managed with daily insulin injections.

Type 2 (T2) diabetes is a common disorder with a prevalence that rises markedly with increasing degrees of obesity and age. The prevalence of T2 diabetes has risen alarmingly in the past decade, in large part linked to the trends in obesity and sedentary lifestyle. Conventional methods of diagnosing early stages of T2 diabetes have limitations. Pre-diabetes and diabetes are widely preventable but are often not diagnosed or diagnosed too late due to the asymptomatic nature of clinical disease progression. If T2 diabetes is detected early, treatments and lifestyle changes can be more effective. It is also possible to prevent onset of the disease.

Embodiments include methods of identifying whether a subject is at risk for developing T2 diabetes (i.e., predisposed to the condition) prior to the occurrence of typical symptoms such as hyperglycemia and/or elevated A1c. Embodiments also include methods of determining the progression (or regression) of T2 diabetes (or pre-diabetes) in a subject. The invention is based on the finding that susceptibility to diabetes can be reliably identified based on particular protein expression profiles with high sensitivity and specificity. The methods described herein can include the combined measurement of at least protein/peptide biomarkers and/or fragments of protein biomarkers referenced in Table 1 from human serum, plasma or a derivative of blood, or blood itself.

TABLE 1
Protein Biomarkers for Prediction/Early Detection of Diabetes

| | biomarker | normal range (serum) | | elevated range |
|---|---|---|---|---|
| 1 | Misfolded Proinsulin | less than 5% of | | greater than 5% |
| | | total proinsulin | | |
| 2 | Correctly Folded | less than 22 | | greater than 22 pmol/L |
| | Proinsulin | pmol/L (fasting) | | |
| 3 | Follistatin | 11.5-13.5 | ng/mL | greater than 14 ng/ml |
| 4 | Hemoglobin A1c (A1c) | less than 5.7% | | greater than 5.7% |
| 5 | C-Reactive Protein | less than 0.9 mg/dL | | greater than 0.9 mg/L |
| | (CRP) | | | |
| 6 | Interleukin 18 (IL-18) | 2,000-3,000 | pg/ml. | greater than 3,000 pg/ml |
| 7 | Interleukin 1 Receptor | 650 | pg/ml | greater than 650 pg/ml |

Antagonist (IL-1RA)

### Proinsulin Hormone

Proinsulin is the precursor of insulin and C-peptide (connecting peptide). Following synthesis, proinsulin is packaged into secretory granules, where it is processed to C-peptide and insulin by prohormone convertases (PC1/3 and PC2) and carboxypeptidase E. Only 1% to 3% of proinsulin is secreted intact. However, because proinsulin has a longer half-life than insulin, circulating proinsulin concentrations are in the range of 5% to 30% of circulating insulin concentrations on a molar basis, with the higher relative proportions seen after meals and in patients with insulin resistance or early type 2 diabetes. Proinsulin can bind to the insulin receptor and exhibits 5% to 10% of the metabolic activity of insulin.

Correctly folded proinsulin hormone is composed of three disulfide bonds, with no incorrect thioester linkages. This marker can serve as a control to evaluate the ratio between misfolded and unfolded proinsulin hormone.

### Misfolded Proinsulin

Misfolded proinsulin can be correlated with progression of T2 diabetes (T2D). Studies have demonstrated that increased proinsulin misfolding via disulfide-linked complexes is an early event associated with beta cell dysfunction that worsens with the onset of prediabetes. Constant demand for insulin prohormone in diabetics can lead to improper tertiary protein folding of the proinsulin during passage through the endoplasmic reticulum. If three critical disulfide bonds are not properly formed, the result is misfolded proinsulin and ultimately less insulin production.

As described below, antibodies can be used to detect (and distinguish) both properly folded and misfolded proinsulin.

### Follistatin

Follistatin, also known as activin-binding protein, is a protein that in humans is encoded by the FST gene. Follistatin is a secreted protein that is expressed in almost all tissues. It is linked to metabolic diseases with elevated plasma levels in patients clearly associated with T2D. Evidence suggests that follistatin has multiple auto- and paracrine functions in various tissues that facilitate binding and neutralization of TGF-β family members. Follistatin is essential for the formation and growth of muscle fibers and is involved in the development of muscle fiber hypertrophy.

Follistatin is expressed in many tissues including endothelial cells, skeletal muscle, pituitary gland and brain, and functions in tissue inflammation and repair. In migrating endothelial cells it is an angiogenic factor that is required for wound healing. It binds to and antagonizes activin-A, a member of the transforming growth factor-β family of growth factors involved in inflammation, fibrosis and cellular proliferation. Follistatin also antagonizes myostatin, a negative regulator of muscle growth and also a member of the transforming growth factor-β superfamily.

### Hemoglobin A1c

As described above, conventional tests often use glycated hemoglobin (HbA1c) for detection of T2 diabetes and pre-diabetes in asymptomatic patients. Although HbA1c can be used to detect diabetes, the levels do not provide the complete picture and diagnosis of diabetes. Embodiments include the use of HbA1c in combination with one or more additional markers such as misfolded proinsulin, correctly folded proinsulin, follistatin, hemoglobin A1c, C-Reactive Protein, Interleukin 18 and Interleukin 1 Receptor Antagonist.

### C-Reactive Protein

C-Reactive Protein (CRP) is an annular (ring-shaped) pentameric protein found in blood plasma, whose circulating concentrations rise in response to inflammation. It is an acute-phase protein of hepatic origin that increases following interleukin-6 secretion by macrophages and T cells. Its physiological role is to bind to lysophosphatidylcholine expressed on the surface of dead or dying cells (and some types of bacteria) in order to activate the complement system via Clq. CRP is synthesized by the liver in response to factors released by macrophages and fat cells (adipocytes). It is a member of the pentraxin family of proteins.

CRP is used mainly as an inflammation marker. Apart from liver failure, there are few known factors that interfere with CRP production. Interferon alpha inhibits CRP production from liver cells which may explain the relatively low levels of CRP found during viral infections compared to bacterial infections.

In patients with type 2 diabetes, low grade inflammation is reflected by increased plasma levels of several biomarkers of inflammation such as C-Reactive Protein (CRP). Small increases in CRP predict the likelihood of developing cardiovascular events both in diabetic and nondiabetic populations. In addition, in apparently healthy subjects, increased levels of CRP predict the risk of developing type 2 diabetes.

Although CRP can be useful to detect diabetes, the levels do not provide the complete picture and diagnosis of diabetes. Embodiments include the use of CRP combined with one or more of the other biomarkers described herein for early prediction of T2 diabetes.

### Interleukin 18

Interleukin-18 (IL18, also known as interferon-gamma inducing factor) is a proinflammatory cytokine. Many cell types, both hematopoietic cells and non-hematopoietic cells, have the potential to produce IL-18. IL-18 is constitutively expressed in non-hematopoietic cells, such as intestinal epithelial cells, keratinocytes, and endothelial cells. IL-18 can modulate both innate and adaptive immunity and its dysregulation can cause autoimmune or inflammatory diseases.

Interleukin-18 (IL-18) is an inflammatory cytokine found to be elevated in type 2 diabetes (T2D) as a part of the chronic low-grade inflammatory process in these states. Embodiments include the use of IL-18 combined with one or more of the other biomarkers described in this application.

### Interleukin 1 Receptor Antagonist

Interleukin 1 Receptor Antagonist (IL-1 RA) is a member of the interleukin 1 cytokine family. IL1 Ra is secreted by various types of cells including immune cells, epithelial cells, and adipocytes, and is a natural inhibitor of the pro-inflammatory effect of IL1β. This protein inhibits the activities of interleukin 1, alpha (IL1A) and interleukin 1, beta (IL1B), and modulates a variety of interleukin 1 related immune and inflammatory responses.

Interleukin-1 receptor antagonist (IL-1 Ra), a natural inhibitor of interleukin-1β, has been shown to improve β-cell function and glycemic control in patients with type 2 diabetes. Studies have shown that individuals who will develop type 2 diabetes are characterized by a complex immune activation that also includes upregulation of the anti-inflammatory cytokine IL-1 Ra.

### Fructosamine/Glycated Hemoglobin

Fructosamines are compounds that result from glycation reactions between a sugar (e.g., fructose or glucose) and a primary amine, followed by isomerization via the Amadori rearrangement. Serum fructosamine represents the total glycated serum proteins, whereas glycated albumin is expressed as a ratio of glycated albumin to total albumin.

Glycated albumin (GA) is the result of non-enzymatic glycation of albumin that occurs in circulating albumin. The rate of glycation of albumin depends on glycemia and on the time that albumin stays in the bloodstream. Because of this, levels of glycated albumin (GA) can reflect short-term glycemia. Levels of GA are not influenced by situations that falsely alter A1C levels (e.g., hemolytic, secondary or iron deficiency anemia, hemoglobinopathies, pregnancy and uremia). Due to the shorter lifespan of albumin in comparison to the traditional biomarkers of glycemic control (e.g., HbA1c), GA can be used as a biomarker of early response to treatments for diabetes.

Embodiments include methods of identifying whether a subject is at risk for developing T2 diabetes (i.e., predisposed to the condition) by utilizing advanced glycation endpoints (AGE). The progressive changes in the ratio between fructosamine (or glycated hemoglobin) and HbA1C can be used as a predictive and prognostic measure to identify patients who have a predisposition to Type 2 Diabetes.

The methods can also be used to determine the progression (or regression) of T2 diabetes (or pre-diabetes). In aspects, the invention is based on the finding that susceptibility to diabetes can be identified based on progressive changes in the ratio between fructosamine and glycated hemoglobin (HbA1C). Alternatively, changes between glycated albumin and glycated hemoglobin (HbA1C) can be used. The methods described herein can include the combined measurement of glycated hemoglobin and HbA1C referenced in Table 2 from human serum, plasma or a derivative of blood, or blood itself.

TABLE 2
Glycation Biomarkers for T2 Diabetes

| | biomarker | normal range (serum) | elevated range |
|---|---|---|---|
| 1 | Glycated Hemoglobin | 11.9-15.8% | greater than 15.8% |
| 2 | Fructosamine | 14% | 17% or higher |
| | | (200-285 μmol/L) | |
| 3 | Hemoglobin | less than 5.7% | greater than 5.7% |
| | A1c (HbA1c) | | |

Accordingly, embodiments also include methods of identifying whether a subject is at risk for developing T2 diabetes (i.e., predisposed to the condition) prior to the occurrence of typical symptoms such as hyperglycemia and/or elevated A1c. The methods can also be used to determine the progression (or regression) of T2 diabetes (or pre-diabetes). In aspects, the invention is based on the finding that susceptibility to diabetes can be reliably identified based on progressive changes in the ratio between fructosamine and glycated hemoglobin (HbA1C). Alternatively, changes between glycated albumin and glycated hemoglobin (HbA1C) can be used with high sensitivity and specificity. The methods described herein can include the combined measurement of at least protein/peptide biomarkers and/or fragments of protein biomarkers referenced in Table 2 from human serum, plasma or a derivative of blood, or blood itself.

### Lipid Intermediary Biomarkers

Lipids have a variety of biological functions in life processes (e.g., cell membrane formation, energy storage and cell signaling, etc.) and they can reflect most metabolic states in health and disease. Recent studies have demonstrated that disorders or abnormalities in lipid metabolism can lead to a variety of human diseases, including diabetes, obesity, atherosclerosis and coronary heart disease. Applicants have discovered that levels of lipid intermediaries can be analyzed and compared for early detection of diabetes.

Embodiments also include the use of the ratio and fold change values of lipid intermediary biomarkers as a predictive and prognostic measures for early detection (and progression) of Type 2 diabetes. The lipid intermediate biomarkers used in these ratio analyses can include protectin D1, lipoxins and maresins. The progressive changes in the ratio and/or fold change values of lipid intermediary biomarkers can be used as a predictive and prognostic measure to identify patients who have a predisposition to Type 2 Diabetes.

Protectin D1 also known as neuroprotectin D1 (when it acts in the nervous system), and abbreviated most commonly as PD1 or NPD1, is a member of the class of specialized proresolving mediators. Specifically, PD1 is an endogenous stereoselective lipid mediator classified as an autocoid protectin. Like other members of this class of polyunsaturated fatty acid metabolites, it possesses strong anti-inflammatory, anti-apoptotic and neuroprotective activity. PD1 is an aliphatic acyclic alkene 22 carbons in length with two hydroxyl groups at the 10 and 17 carbon positions and one carboxylic acid group at the one carbon position.

A lipoxin (LX or Lx), an acronym for lipoxygenase interaction product, is a bioactive autacoid metabolite of arachidonic acid made by various cell types. They are categorized as nonclassic eicosanoids and members of the specialized pro-resolving mediators (SPMs) family of polyunsaturated fatty acid (PUFA) metabolites. Like other SPMs, LXs form during, and then act to resolve, inflammatory responses.

Maresins are lipids produced by macrophages that contribute to wound repair and reduce nerve sensitivity to painful stimuli. Maresins are biosynthesized by macrophages that are critically important in restoring tissue homeostasis after inflammation. Maresin 1 (MaR1 or 7R,14S-dihydroxy-4Z,8E,10E,12Z,16Z,19Z-docosahexaenoic acid) is a macrophage-derived mediator of inflammation resolution coined from macrophage mediator in resolving inflammation. Maresin 1, and more recently defined maresins, are 12-lipoxygenase-derived metabolites of the omega-3 fatty acid, docosahexaenoic acid (DHA), that possess potent anti-inflammatory, pro-resolving, protective, and pro-healing properties similar to a variety of other members of the specialized proresolving mediators (SPM) class of polyunsaturated fatty acid (PUFA) metabolites.

TABLE 3
Lipid Intermediary Biomarkers for T2 Diabetes

| | | normal range | |
|---|---|---|---|
| | biomarker | (serum) | elevated range |
| 1 | Protectin D1 | 50-200 pg/mL | 250 pg/mL or higher |
| 2 | Lipoxins | 50-200 pg/mL | 250 pg/mL or higher |
| 3 | Maresins | 50-200 pg/mL | 250 pg/mL or higher |

Accordingly, embodiments also include methods of identifying whether a subject is at risk for developing T2 diabetes (i.e., predisposed to the condition) prior to the occurrence of typical symptoms such as hyperglycemia and/or elevated A1c. The methods can also be used to determine the progression (or regression) of T2 diabetes (or pre-diabetes). In aspects, the invention is based on the finding that susceptibility to diabetes can be reliably identified based on progressive changes in the ratio between lipid intermediaries. The methods described herein can include the combined measurement of at least a lipid intermediary biomarker and/or fragment thereof as referenced in Table 3 from human serum, plasma or a derivative of blood, or blood itself.

### Method of Testing Biomarkers

The biomarkers identified in Table 1 can be identified and their levels determined using antibody-based methods, such as, an enzyme-linked immunosorbent assay (ELISA), a radioimmunoassay (RIA) or a lateral flow immunoassay (LFA). In one embodiment, the method uses a lateral flow assay that can detect protein biomarkers at low levels. The levels of protein can also be determined by analyzing the amount of an indicator of fluorescence.

LFA-based tests are well known in the art. A typical LFA can include the following components: sample pad, conjugate release pad, membrane with immobilized antibodies and adsorbent pad. The components of the strip are usually fixed to an inert backing material. A biological sample is applied to a portion of the strip (i.e., sample pad) of a lateral flow assay, resulting in the accumulation of a labelled binding reagent, in an analyte concentration-dependent manner (proportional or inversely proportional) at a detection zone on the test strip. The test strip can then be analyzed to detect/quantify the amount of target analytes (i.e., biomarkers) by assay reading components mounted on the PCBA. A microprocessor, ASIC or the like can analyze and interpret the readings and display the assay results to a user and/or health care provider.

In one embodiment, the method includes a multi-line lateral flow test strip that uses a sandwich-antibody capture technique to quantify the biomarkers identified in Table 1 and/or Table 2. The method can use an up-converting phosphor (UCP) detection system that provides high sensitivity, zero biological background florescent and results in ten-fold dynamic range increase over other antibody detection systems. Further, the UCP detection system is quantitative and lasts for over twenty years.

The amount of an analyte present in a sample can be determined in absolute terms (e.g., in terms of a numerical value per unit volume) or in relative terms (e.g., by reference to a predetermined threshold). In particular, the “relative amount of one or more biomarkers” is not intended to mean that the concentrations of different biomarkers in a sample are compared with one another, but rather that the concentration of one or more such analytes may be compared to a predetermined threshold.

The measurement of the assay and/or interpretation of the assay result can include one or more data processing steps, in which assay data are subjected to one or more computations or other type of processing. Such processing can be performed by a digital electronic device such as a microprocessor or the like, which will typically form part of an extrinsic, or an integral, assay result reader. For example, the data processing can include the calculation of a ratio.

One or more of the biomarkers can be used in a method of predicting the susceptibility that a subject will develop an ailment (e.g., T1 or T2 diabetes) or determining the progression of a test subject with T1 or T2 diabetes. In this manner, one biomarker or a combination of 2, 3, 4, 5, 6, 7, 8, 9, 10, 11, 12, 13, 14, 15, 16, 17 and/or 18 biomarkers can be used in the method. In this manner, at least one biomarker or a combination of at least 2, 3, 4, 5, 6, 7, 8, 9, 10, 11, 12, 13, 14, 15, 16, 17 and/or 18 biomarkers can be used in the method.

In this manner, no more than one biomarker or a combination of no more than 2, 3, 4, 5, 6, 7, 8, 9, 10, 11, 12, 13, 14, 15, 16, 17 and/or 18 biomarkers can be used in a method of diagnosing an ailment or determining a prognosis of a test subject with an ailment. In this manner, about one biomarker or a combination of about 2, 3, 4, 5, 6, 7, 8, 9, 10, 11, 12, 13, 14, 15, 16, 17 and/or 18 biomarkers can be used in a method of diagnosing an ailment or determining a prognosis of a test subject with an ailment (e.g., T1 or T2 diabetes).

In a first step, the expression levels of one or more proteins are measured in plasma samples from subjects with an ailment (e.g., T1 or T2 diabetes). In an embodiment, the expression levels of one biomarker or a combination of 2, 3, 4, 5, 6, 7, 8, 9, 10, 11, 12, 13, 14, 15, 16, 17 and/or 18 biomarkers can be used to generate a footprint or signature for subsequent diagnosis of patients. In an embodiment, the expression levels of at least one biomarker or a combination of at least 2, 3, 4, 5, 6, 7, 8, 9, 10, 11, 12, 13, 14, 15, 16, 17 and/or 18 biomarkers can be used to generate a footprint or signature for subsequent diagnosis of patients.

In an embodiment, the expression levels of no more than one biomarker or a combination of no more than 2, 3, 4, 5, 6, 7, 8, 9, 10, 11, 12, 13, 14, 15, 16, 17 and/or 18 biomarkers are used to generate a footprint or signature for subsequent diagnosis of patients. In an embodiment, the expression levels of about one biomarker or a combination of about 2, 3, 4, 5, 6, 7, 8, 9, 10, 11, 12, 13, 14, 15, 16, 17 and/or 18 biomarkers can be used to generate a footprint or signature for subsequent diagnosis of patients.

Next, expression levels of the same proteins are measured in plasma, blood or tissue samples from healthy subjects. This is used as a control. Thereafter, samples from healthy patients can be compared to identifying proteins that have altered levels of expression in the plasma samples from the subjects with an ailment. A biomarker fingerprint or signature can be created from the proteins with altered levels of expression. This can be used for diagnosing or determining the prognosis of an ailment in the test subject by comparing levels of proteins from plasma of the test subject. Conventional statistical analysis can be used to determine, for example, confidence levels.

A biomarker that is “upregulated” generally refers to an increase in the level of expression in response to a given treatment or condition. A biomarker that is “downregulated” generally refers to a decrease in the level of expression of the biomarker in response to a given treatment or condition. In some situations, the biomarker level can remain unchanged upon a given treatment or condition. A biomarker from a patient sample can be “upregulated,” i.e., the level can be increased, for example, by about 5%, about 10%, about 20%, about 30%, about 40%, about 50%, about 60%, about 70%, about 80%, about 90%, about 100%, about 200%, about 300%, about 500%, about 1,000%, about 5,000% or more compared to a reference level. Alternatively, a biomarker can be “downregulated,” i.e., the level can be decreased, for example, by about 99%, about 95%, about 90%, about 80%, about 70%, about 60%, about 50%, about 40%, about 30%, about 20%, about 10%, about 5%, about 2%, about 1% or less compared to a reference level.

In embodiments, a subject is treated with one or more medicaments upon determining their predisposition to T1 or T2 diabetes. The medicament can also be administered to slow/inhibit progression of the disease. Medicaments can include, for example, metformin, repaglinide, albiglutide, dulaglutide, exenatide (and exenatide extended-release), liraglutide and semaglutide. In some embodiments, a subject is administered insulin for treatment. Treatment can also include lifestyle changes (e.g., diet and exercise). Immunomodulating agents can be administered to prevent (or ameliorate) progression of T1 diabetes (or transition to T1 diabetes). Such agents include, specific immunomodulating agents (e.g., monoclonal antibodies, cytokines and vaccines) and nonspecific immunomodulating agents (e.g., BCG and levamisole, steroids, Cyclosporine A, tacrolimus, azathioprine and T cell inhibitors).

### Diagnostic Kit for Diabetes Screening

The following working example is based on configurations described above. Embodiments of the invention can be compiled into a diagnostic kit for diagnosing an ailment such as a senescence-associated disease or disorder. The kit can identify one or more target cells that have the biomarkers for ailment in plasma from a test subject.

For example, immunoassay kits can be used as described herein. Such kits can include, (a) antibodies having binding specificity for the polypeptides used in the diagnosis of a pre-diabetes or diabetes; and (b) anti-antibody immunoglobulins. This immunoassay kit can be utilized for the practice of the various methods provided herein. The antibodies and the anti-antibody immunoglobulins can be provided in an amount of about 0.001 mg to 100 grams, and more preferably about 0.01 mg to 1 gram. The anti-antibody immunoglobulin may be a polyclonal immunoglobulin (monoclonal, recombinant or cocktail thereof), protein A or protein G or functional fragments thereof, which may be labeled prior to use by methods known in the art. In embodiments, the immunoassay kit includes two, three, four, five, six or seven of: antibodies that specifically bind to each of the following proteins: misfolded proinsulin, correctly folded proinsulin, follistatin and hemoglobin A1c, C-Reactive Protein, Interleukin 18 and Interleukin 1 Receptor Antagonist.

The kit can also include reagents that can be used to identify variations in expression levels of one or more proteins in a sample from a test subject. The expression levels of the proteins can be used in a comparison/analysis of test samples with a fingerprint indicative of the susceptibility of developing T2 diabetes. Similarly, the expression levels can be used for determining the progression (or regression) of T2 diabetes in a subject.

An indicator compound can be used to determine the amount of each biomarker. The indicator compound can be any compound, chemical, or biological component which may interact with a target or byproduct of a target. For example, an indicator compound may comprise an antibody, a reactive chemical compound, a labeled molecule, or any combination thereof. Antibodies used in an embodiment of the present disclosure may be monoclonal or polyclonal and derived from any species (e.g., human, rat, mouse, rabbit, pig). Further, indicator molecules may be aptamers, proteins, peptides, small organic molecules, natural compounds, non-peptide polymers, MHC multimers (including MHC-dextramers, MHC-tetramers, MHC-pentamers and other MHC-multimers), or any other molecules that specifically and efficiently bind to other molecules.

Labeled molecules, for use as indicator compounds, may be any molecule that absorbs, excites, or modifies radiation, such as the absorption of light (e.g., dyes and chromophores) and the emission of light after excitation (fluorescence from fluorochromes). Additionally, labeled molecules may have an enzymatic activity, by which it catalyzes a reaction between chemicals in the near environment of the labeling molecules, producing a signal which include production of light (chemi-luminescence) or precipitation of chromophors, dyes, or a precipitate that can be detected by an additional layer of detection molecules.

The indictor signal in at least one embodiment of the present disclosure may comprise any detectable signal, including but not limited to, color change, fluorescence, and chemical/structural change of the target (and/or indicator compound) so as to be amenable to reacting with a secondary detection marker. Further, the detection of a signal may involve a secondary reactive molecule.

The target of an exemplary indicator compound of the present disclosure may be any diagnostic marker or diagnostic condition for a disease state (or pre-disease state) of an individual. For example, the disease state may be diabetes, pre-diabetes or metabolic syndrome.

A kit can include one biomarker or a combination of 2, 3, 4, 5, 6, 7, 8, 9, 10, 11, 12, 13, 14, 15, 16, 17 and/or 18 biomarkers disclosed herein. The skilled artisan will appreciate that the number of biomarkers may be varied without departing from the nature of the disclosure, and thus other combinations of biomarkers are also encompassed by the disclosure. The skilled artisan will know which one biomarker or a combination of 2, 3, 4, 5, 6, 7, 8, 9, 10, 11, 12, 13, 14, 15, 16, 17 and/or 18 biomarkers to use. It is also contemplated that additional medical information can be used in determining the susceptibility and/or progression of T2 diabetes in a subject.

In a specific embodiment, a kit includes the one biomarker or a combination of 2, 3, 4, 5, 6, 7, 8, 9, 10, 11, 12, 13, 14, 15, 16, 17 and/or 18 biomarkers disclosed herein. The kit can further optionally include instructions for use. The kit can further optionally include (e.g., comprise, consist essentially of, consist of) tubes, applicators, vials or other storage containers with the above-mentioned biomarker and/or vials containing one or more of the biomarkers. In an embodiment, each biomarker is in its own tube, applicator, vial or storage container or 2, 3, 4, 5, 6, 7, 8, 9, 10, 11, 12, 13, 14, 15, 16, 17 and/or 18 biomarkers are in a tube, applicator, vial or storage container.

The kits, regardless of type, will generally include one or more containers into which the one biomarker or a combination of 2, 3, 4, 5, 6, 7, 8, 9, 10, 11, 12, 13, 14, 15, 16, 17 and/or 18 biomarkers are placed and, preferably, suitably aliquoted. The components of the kits may be packaged either in aqueous media or in lyophilized form.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Early Detection of T2 Diabetes

In this example, a 30-year-old male visits a clinic for a regular checkup. A healthcare provider wishes to determine the subject's susceptibility to developing T2 diabetes. The test includes an immunochromatographic membrane assay that uses antibodies to detect each biomarker (i.e., misfolded proinsulin, correctly folded proinsulin, follistatin, hemoglobin A1c, C-Reactive Protein, Interleukin 18 and Interleukin 1 Receptor Antagonist). The amount of each biomarker is analyzed and increased amounts of each indicate that the subject is at risk of developing T2 diabetes.

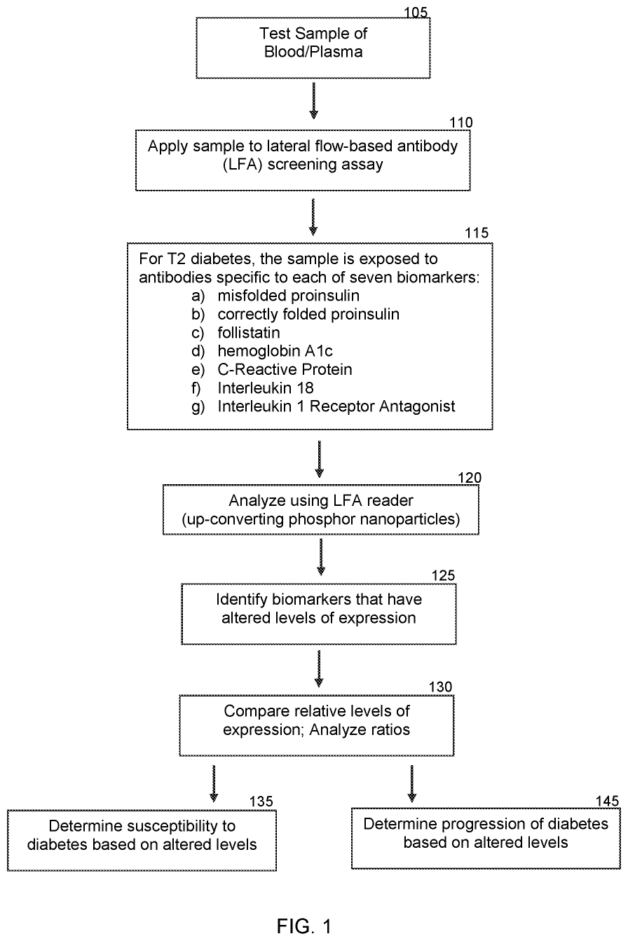

FIG. 1 is a flow chart detailing steps in a method of measuring the biomarkers. A blood of plasma sample (typically a few drops) is obtained from a test subject (105) and applied to the sample portion of a test strip (110). The sample is incubated at room temperature (e.g., five to ten minutes) to allow the sample to diffuse across test strip passing through the conjugate pad into the nitrocellulose membrane and then onto the absorbent pad (115). In this example, the antibodies use fluorescence (i.e., up-converting phosphor (UCP) detection).

The amount of each biomarker is determined by the relative levels of fluorescence. The test strip is analyzed using a lateral flow analysis (LFA) reader (120). The values are compared to control samples to determine whether biomarker levels are normal or elevated. In this example, the following results are obtained:

TABLE 4
Biomarker Levels

| | Biomarker | Control levels | | Test levels | |
|---|---|---|---|---|---|
| 1 | Misfolded Proinsulin | 5% of total | | 7.5% of total | |
| | | proinsulin | | proinsulin | |
| 2 | Correctly Folded | less than 20 | | 25 | pmol/L |
| | Proinsulin | pmol/L (fasting) | | | |
| 3 | Follistatin | 12.5 | ng/mL | 16.5 | ng/mL |
| 4 | Hemoglobin A1c | 5.0% | | 6.5% | |
| 5 | C-Reactive Protein | <0.9 | mg/dL | 1 | mg/dL |
| 6 | Interleukin 18 | 2,000-3,000 | pg/mL | 3,500 | pg/mL |

| | | | | |
|---|---|---|---|---|
| 7 | Interleukin 1 | mean of 654 pg/ml | 700 | pg/ml |

Receptor Antagonist

As noted in Table 2, each of the biomarkers is elevated in the test sample (125). Based on these values, the healthcare professional determines that the subject is susceptible to developing T2 diabetes (130). Additional biomedical information can also be considered in evaluating the patient. The subject is advised to exercise, maintain a healthy BMI and stick to a diet with a low glycemic index. One or more medicaments can be administered to the subject (e.g., metformin). The subject is tested periodically (e.g., semi-annually) to monitor changes in biomarker levels.

### Example 2

### Monitoring Progression of T2 Diabetes

In this example, a 40-year-old female visits a clinic for follow up. Previous lab work has indicated T2 diabetes based on elevated levels of fasting glucose and an elevated A1c. Since her previous visit, the patient has regularly exercised, reduced her weight/BMI and stuck to a diet with a low glycemic index.

A healthcare provider wishes to determine the subject's progression of T2 diabetes (145). To do so, the provider compares the change in biomarker levels over time. Levels are obtained on a first visit to the clinic (D1) and again after six months (D2). The amount of each biomarker is determined as described above. The values are compared to control samples to determine whether biomarker levels are normal or elevated. In this example, the following results are obtained:

TABLE 5
Change in Biomarker Levels

| | Biomarker | Test levels (D1) | | Test levels (D2) | |
|---|---|---|---|---|---|
| 1 | Misfolded Proinsulin | 7.5% of total proinsulin | | 7.0% of total proinsulin | |
| 2 | Correctly Folded | 25 | pmol/L | 23 | pmol/L |
| | Proinsulin | | | | |
| 3 | Follistatin | 16.5 | ng/mL | 16.0 | ng/mL |
| 4 | Hemoglobin A1c | 6.5% | | 6.4% | |
| 5 | C-Reactive Protein | 0.95 | mg/dL | 0.88 | mg/dL |
| 6 | Interleukin 18 | 2,750 | pg/mL | 2,750 | pg/mL |
| 7 | Interleukin 1 | 750 | pg/ml | 700 | pg/ml |

Receptor Antagonist

As noted in Table 5, each of the biomarkers is higher in the D1 test sample. Based on the change in values, the healthcare professional determines that there is no progression in T2 diabetes. Lower levels of each biomarker can indicate some regression of the disease. Additional biomedical information can also be considered. The subject is advised to exercise, maintain a healthy BMI and stick to a diet with a low glycemic index. The subject is tested periodically (e.g., semi-annually) to monitor changes in biomarker levels.

### Example 3

### Prediction of T1 Diabetes

In this example, a 20-year-old male visits a clinic for a regular checkup. A healthcare provider wishes to determine the subject's susceptibility to developing T1 diabetes. The test includes an immunochromatographic membrane assay that uses antibodies to detect each biomarker (i.e., GAD65, ICA, IAA, ZnT8, IA-2A and C-reactive protein). The amount of each biomarker is analyzed and increased amounts of each indicate that the subject is at risk of developing T1 diabetes.

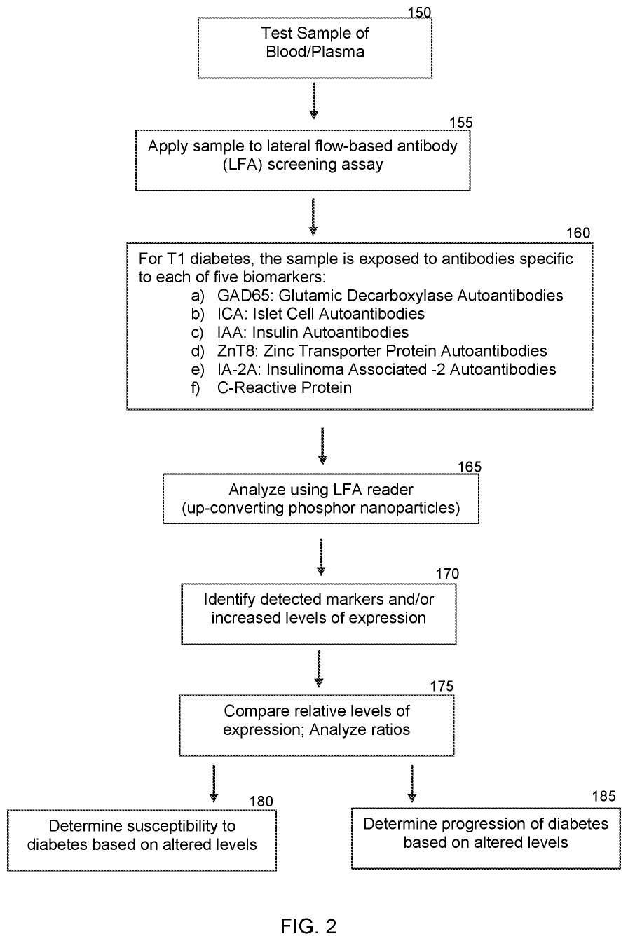

FIG. 2 is a flow chart detailing steps in a method of measuring the biomarkers. A blood of plasma sample (typically a few drops) is obtained from a test subject (150) and applied to the sample portion of a test strip (155). The sample is incubated at room temperature (e.g., five to ten minutes) to allow the sample to diffuse across test strip passing through the conjugate pad into the nitrocellulose membrane and then onto the absorbent pad (160). In this example, the antibodies use fluorescence (i.e., up-converting phosphor (UCP) detection).

The amount of each biomarker is determined by the relative levels of fluorescence. The test strip is analyzed using a lateral flow analysis (LFA) reader (165). The values are compared to control samples to determine whether biomarker levels are normal or elevated. In this example, the following results are obtained:

TABLE 6
Biomarker Levels

| | Biomarker | Test levels |
|---|---|---|
| 1 | GAD65: Glutamic | detected |
| | Decarboxylase | |
| | Autoantibodies | |
| 2 | ICA: Islet Cell Autoantibodies | detected |
| 3 | IAA: Insulin Autoantibodies | undetected |
| 4 | ZnT8: Zinc Transporter | detected |
| | Protein Autoantibodies | |
| 5 | IA-2A: Insulinoma | undetected |
| | Associated-2 | |
| | Autoantibodies | |
| 6 | C-Reactive Protein | detected |

As noted in Table 6, four of six of the markers were detected in the test sample (125). Based on these values, the healthcare professional determines that the subject is susceptible to developing T1 diabetes (180). Additional biomedical information can also be considered in evaluating the patient. The subject is advised to exercise, maintain a healthy BMI and stick to a diet with a low glycemic index. One or more medicaments can be administered to the subject (e.g., metformin). The subject is tested periodically (e.g., semi-annually) to monitor changes in biomarker levels.

### Example 4

### Predicting T2 Diabetes (Ratio of Glycated Albumin to HbA1C)

Embodiments also include a method of predicting or identifying a predisposition to diabetes. The method can include detecting changes in the ratio between fructosamine (i.e., glycated albumin) and HbA1C (i.e., glycated hemoglobin). Alternatively, the method can include detecting changes in the ratio between glycated albumin and HbA1C.

In this example, a 40-year-old male visits a clinic for a regular checkup. The patient had previously been screened for T2 diabetes because of risk factors (i.e., obesity and family history). He was counseled on diet and lifestyle changes. A healthcare provider wishes to determine whether the patient is at risk, despite improvements in his diet and lifestyle. The test includes an immunochromatographic membrane assay that uses antibodies to detect each biomarker (i.e., glycated albumin and HbA1c). The amount of each biomarker is analyzed and compared.

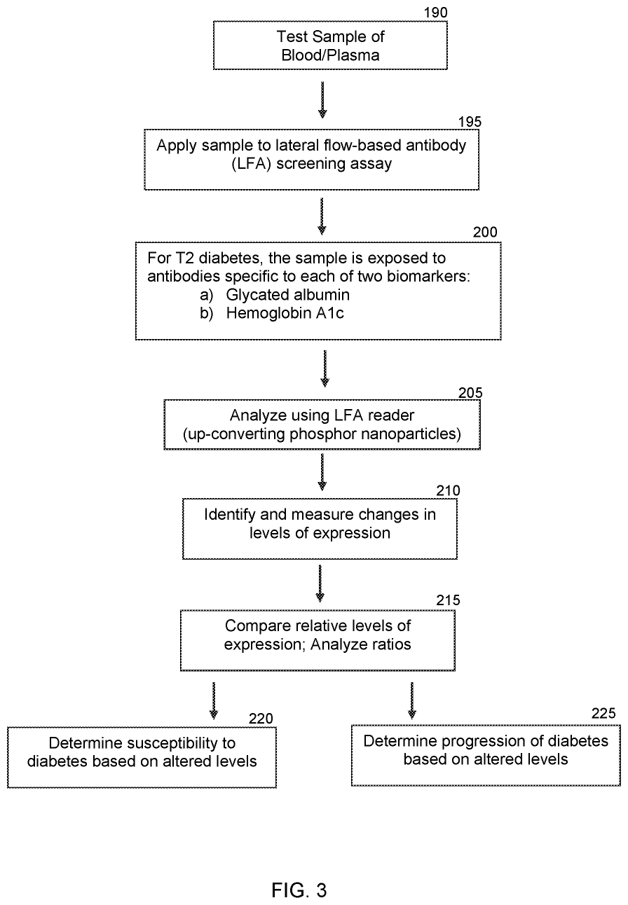

FIG. 3 is a flow chart detailing steps in a method of measuring the biomarkers. A blood of plasma sample (typically a few drops) is obtained from a test subject (190) and applied to the sample portion of a test strip (195). The sample is incubated at room temperature (e.g., five to ten minutes) to allow the sample to diffuse across test strip passing through the conjugate pad into the nitrocellulose membrane and then onto the absorbent pad (200). In this example, the antibodies use fluorescence (i.e., up-converting phosphor (UCP) detection).

The amount of each biomarker is determined by the relative levels of fluorescence. The test strip is analyzed using a lateral flow analysis (LFA) reader (205). The values are compared to those from a previous test (one year earlier) to compare levels. In this example, the following results are obtained:

TABLE 7
Glycation Biomarkers for T2 Diabetes

| | | normal | |
|---|---|---|---|
| | biomarker | range (serum) | Test levels |
| 1 | Glycated | 11.9-15.8% | 17.5% |
| | Hemoglobin | | |
| 2 | Hemoglobin | less than 5.7% | 6.5% |
| | A1c (HbA1c) | | |

As noted in Table 7, the levels of markers were slightly elevated. The healthcare provider calculates a ratio of GH/HbA1c of 2.69. Based on these values, the healthcare professional determines that the subject is susceptible to developing T2 diabetes (220). The subject is advised to exercise, maintain a healthy BMI and stick to a diet with a low glycemic index. One or more medicaments can be administered to the subject (e.g., metformin). The subject is tested periodically (e.g., semi-annually) to monitor changes in biomarker levels.

### Example 5

### Early Detection of T2 Diabetes (Lipid Intermediary Biomarkers)

Embodiments also include a method of identifying/diagnosing an ailment (e.g., T2 diabetes) or determining a prognosis of the ailment. The method can include detecting changes in the ratio of lipid intermediary biomarkers. The lipid intermediate biomarkers can include Protectin D1, Lipoxins, and Maresins, among others.

In this example, a 40-year-old female visits a clinic for a regular checkup. The patient had previously been screened for T2 diabetes because of risk factors (i.e., obesity and family history). A healthcare provider wishes to determine whether the patient remains at risk, despite improvements in her diet and lifestyle. The test includes quantifying each biomarker of three markers (i.e., Protectin D1, Lipoxins, and Maresins).

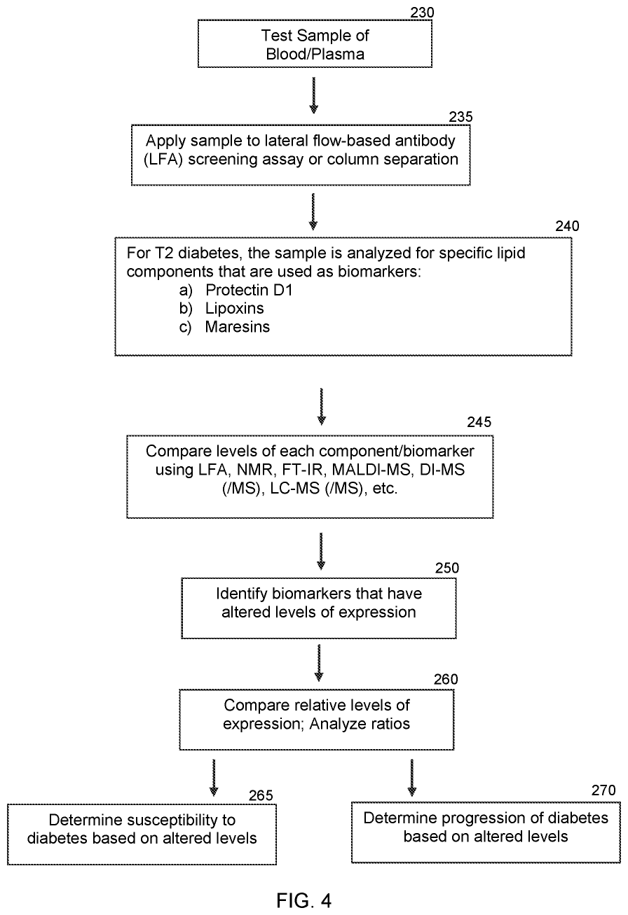

FIG. 4 is a flow chart detailing steps in a method of measuring the biomarkers. A blood plasma sample is obtained from a test subject (230) and applied to the sample portion of a test strip (235). The sample is incubated at room temperature (e.g., five to ten minutes) to allow the sample to diffuse across test strip passing through the conjugate pad into the nitrocellulose membrane and then onto the absorbent pad. In this example, the antibodies use fluorescence (i.e., up-converting phosphor (UCP) detection).

The amount of each biomarker is determined by the relative levels of fluorescence. The test strip is analyzed using a lateral flow analysis (LFA) reader (245). In this example, the following results are obtained:

TABLE 7
Lipid Biomarkers

| | | normal | |
|---|---|---|---|
| | biomarker | range (serum) | Test levels |
| 1 | Protectin D1 | 50-200 pg/mL | 250 pg/mL |
| 2 | Lipoxins | 50-200 pg/mL | 215 pg/mL |
| 3 | Maresins | 50-200 pg/mL | 275 pg/mL |

As noted in Table 7, the levels of markers were slightly elevated. Based on these values, the healthcare professional determines that the subject is susceptible to developing T2 diabetes (265). The subject is advised to exercise, maintain a healthy BMI and stick to a diet with a low glycemic index. The subject is tested periodically (e.g., semi-annually) to monitor changes in biomarker levels.

### Example 6

### Early Detection of T2 Diabetes

In this example, a 65-year-old male visits a clinic for a regular checkup. The patient had previously been screened for T2 diabetes because of risk factors (i.e., age, obesity and family history). His HbA1C level was 5.9% (slightly elevated). A healthcare provider wishes to determine whether the patient has a predisposition to T2 diabetes, despite improvements in his diet and lifestyle. The test includes quantifying each of four markers (i.e., misfolded proinsulin, correctly folded insulin, follistatin and HbA1c).

The amount of each biomarker is determined by the relative levels of fluorescence. The test strip is analyzed using a lateral flow analysis (LFA) reader. In this example, the following results are obtained:

TABLE 8
Biomarker Levels

| | Biomarker | Control levels | Test levels |
|---|---|---|---|
| 1 | Misfolded | 5% of total | 6.5% of total |
| | Proinsulin | proinsulin | proinsulin |
| 2 | Correctly Folded | less than 20 | 22.5 pmol/L |
| | Proinsulin | pmol/L (fasting) | |
| 3 | Follistatin | 12.5 ng/mL | 14.0 ng/mL |
| 4 | Hemoglobin A1c | 5.0% | 6.2% |

As noted in Table 8, the levels of markers were slightly elevated. Based on these values, the healthcare professional determines that the subject is susceptible to developing T2 diabetes. The subject is advised to exercise, maintain a healthy BMI and stick to a diet with a low glycemic index. The subject is tested periodically (e.g., semi-annually) to monitor changes in biomarker levels.

In one embodiment, the methods disclosed herein are capable of determining whether a subject is, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% likely to become T2 diabetic.

In one embodiment, the methods disclosed herein are capable of determining that a subject is pre-diabetic by e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% earlier than a conventional A1c test.

In one embodiment, the methods disclosed herein are capable of reducing the likelihood that an individual will become diabetic by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%.

In embodiments, a ratio of glycated albumin to hemoglobin A1c (GH/HbA1c) indicates a predisposition to diabetes. In aspects, the ratio is at least 0.1, at least 0.2, at least 0.4, at least 0.6, at least 0.8, at least 1.0, at least 1.2, at least 1.4, at least 1.8, at least 2.0, at least 2.2, at least 2.4, at least 2.6, at least 2.8, at least 3.0, at least 3.2, at least 3.4, at least 3.6, at least 3.8, at least 4.0 or higher.

In embodiments, a ratio of glycated albumin to hemoglobin A1c (GH/HbA1c) indicates a predisposition to diabetes. In aspects, the ratio is more than 0.1, more than 0.2, more than 0.4, more than 0.6, more than 0.8, more than 1.0, more than 1.2, more than 1.4, more than 1.8, more than 2.0, more than 2.2, more than 2.4, more than 2.6, more than 2.8, more than 3.0, more than 3.2, more than 3.4, more than 3.6, more than 3.8, more than 4.0 or higher.

In one embodiment, the methods disclosed herein are capable of predicting that a subject will become diabetic at least six months before conventional methods. In aspects, the methods disclosed can predict a subject will become diabetic at least nine months, at least one year, at least two years, at least three years, at least five years, at least seven years, at least ten years, at least twelve years, at least fifteen years of at least twenty years before conventional methods (e.g., elevated HbA1c) or showing signs/symptoms of diabetes.

In aspects, the methods disclosed can predict a subject will become diabetic about nine months, about one year, about two years, about three years, about five years, about seven years, about ten years, about twelve years, about fifteen years of about twenty years before conventional methods (e.g., elevated HbA1c) or showing signs/symptoms of diabetes.

In aspects, the methods disclosed can predict a subject will become affected by one or more of prediabetes, gestational diabetes, metabolic syndrome, insulin resistance, glucose intolerance, glucose non-responsiveness, Type 1 diabetes, Type 1.5 diabetes and/or Type 2 diabetes.

In aspects, the methods disclosed can predict a subject will become affected by one or more of Group 1, Group 2, Group 3, Group 4 or Group 5 diabetes.

Determining if one is predisposed to diabetes or predicting that he/she will suffer from diabetes can include considering additional biomedical information as well as historical data (i.e., data collected about past events and circumstances pertaining to a particular patient or similar patients). In aspects, the methods disclosed can be used to determine a diabetes risk score. It can also be used in conjunction with a diabetes risk score (e.g., as determined by evaluation by a health care professional).

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1.-5. (canceled)

6. A method of monitoring the progression of diabetes in a subject, the method comprising steps of:
a) detecting levels of two or more biomarkers in a sample at a first time point from the subject,
b) detecting levels of the two or more biomarkers in a sample at a second time point from the subject,
c) comparing the levels of the two or more biomarkers from the first time point to the second time point,
d) identifying progression of diabetes based on a change in levels of the two or more biomarkers, and
e) treating the patient to reduce or slow the progression of diabetes.

7. The method of claim 6, wherein the two or more biomarkers are selected from misfolded proinsulin, correctly folded proinsulin, follistatin, hemoglobin A1c, C-Reactive Protein, Interleukin 18 and Interleukin 1 Receptor Antagonist.

8. The method of claim 6, wherein the two or more biomarkers are selected from protectin D1, a lipoxin and a maresin.

9. The method of claim 6, wherein the two or more biomarkers are selected from glutamic decarboxylase autoantibodies, islet cell autoantibodies, insulin autoantibodies, zinc transporter protein autoantibodies, insulinoma associated-2 autoantibodies and C-reactive protein.

10. The method of claim 6, wherein the two or more biomarkers are selected from misfolded proinsulin, correctly folded proinsulin, follistatin, hemoglobin A1c, C-Reactive Protein, Interleukin 18 and Interleukin 1 Receptor Antagonist, glutamic decarboxylase autoantibodies, islet cell autoantibodies, insulin autoantibodies, zinc transporter protein autoantibodies, insulinoma associated-2 autoantibodies, C-reactive protein, protectin D1, a lipoxin and a maresin.

11. The method of claim 6, wherein the diabetes is Type 1 diabetes, Type 1.5 diabetes and/or Type 2 diabetes.

12. The method of claim 6, wherein the diabetes is Group 1, Group 2, Group 3, Group 4 or Group 5 diabetes.

13. The method of claim 6, wherein the step of treating the patient comprises one or more of exercising, losing weight, maintaining a healthy body mass index (BMI), dieting and administering a medicament.

14. The method of claim 13, wherein the medicament is one or more of metformin, repaglinide, albiglutide, dulaglutide, exenatide, extended-release exenatide, liraglutide, semaglutide and insulin.

15. A method of ameliorating or preventing an ailment in a subject, the method comprising steps of:
a) detecting levels of two or more biomarkers in a sample from the subject,
b) identifying a predisposition to the ailment based on the presence of elevated levels of the two or more biomarkers,
c) treating the subject to prevent or ameliorate the ailment.

16. The method of claim 15, wherein the ailment is prediabetes, metabolic syndrome, insulin resistance, glucose intolerance, glucose non-responsiveness, Type 1 diabetes, Type 1.5 diabetes and/or Type 2 diabetes.

17. The method of claim 15, wherein the step of treating the subject comprises one or more of exercising, losing weight, maintaining a healthy body mass index (BMI), dieting and administering a medicament.

18. The method of claim 17, wherein the medicament is one or more of metformin, repaglinide, albiglutide, dulaglutide, exenatide, extended-release exenatide, liraglutide, semaglutide and insulin.

19. The method of claim 15, wherein the two or more biomarkers are selected from misfolded proinsulin, correctly folded proinsulin, follistatin, hemoglobin A1c, C-Reactive Protein, Interleukin 18 and Interleukin 1 Receptor Antagonist.

20. The method of claim 15, wherein the two or more biomarkers are selected from protectin D1, a lipoxin and a maresin.

21. The method of claim 15, wherein the two or more biomarkers are selected from glutamic decarboxylase autoantibodies, islet cell autoantibodies, insulin autoantibodies, zinc transporter protein autoantibodies, insulinoma associated-2 autoantibodies and C-reactive protein.

22. The method of claim 15, wherein the two or more biomarkers are glycated albumin and hemoglobin A1c.

23. The method of claim 15, wherein the two or more biomarkers are selected from misfolded proinsulin, correctly folded proinsulin, follistatin, hemoglobin A1c, C-Reactive Protein, Interleukin 18 and Interleukin 1 Receptor Antagonist, glutamic decarboxylase autoantibodies, islet cell autoantibodies, insulin autoantibodies, zinc transporter protein autoantibodies, insulinoma associated-2 autoantibodies, C-reactive protein, protectin D1, a lipoxin and a maresin.

24. A method of detecting a predisposition to an ailment in a subject, the method comprising steps of:
a) detecting levels of a first biomarker and a second biomarker in a sample from the subject,
b) calculating a ratio of the first biomarker and the second biomarker,
c) identifying the predisposition to the ailment based on the ratio,
d) treating the subject to prevent or ameliorate the ailment,
wherein the first biomarker is fructosamine or glycated albumin and the second biomarker is hemoglobin A1c, and
wherein the ailment is prediabetes, metabolic syndrome, insulin resistance, glucose intolerance, glucose non-responsiveness and/or Type 2 diabetes.

25.-64. (canceled)

65. The method of claim 24, wherein the sample is blood, urine or saliva.
